(19) United States
(12) Patent Application Publication
(10) Pub. No.: US 2025/0195351 A1
(43) Pub. Date: Jun. 19, 2025

(54) BRIGHTENING COMPOSITION AND SKIN BRIGHTENING METHOD USING THE SAME

(71) Applicant: AMOREPACIFIC CORPORATION, Seoul (KR)

(72) Inventors: Hyunjung CHOI, Yongin-si (KR); Soo Jin LEE, Yongin-si (KR); Wonseok PARK, Yongin-si (KR); Heungsoo BAEK, Yongin-si (KR); Yong Deog HONG, Yongin-si (KR)

(73) Assignee: AMOREPACIFIC CORPORATION, Seoul (KR)

(21) Appl. No.: 18/852,151

(22) PCT Filed: Mar. 29, 2023
(86) PCT No.: PCT/KR2023/004141

§ 371 Date: Sep. 27, 2024

(30) Foreign Application Priority Data
Mar. 29, 2022 (KR) 10-2022-0039137
Mar. 28, 2023 (KR) 10-2023-0040647

(51) Int. Cl. A61K 8/35; A61Q 19/02

(52) U.S. Cl. CPC A61K 8/35; A61Q 19/02

(57) ABSTRACT

Provided are a brightening cosmetic composition including a compound represented by Chemical Formula 1 as an active ingredient, and a skin brightening method including applying an effective amount of the composition to the skin:

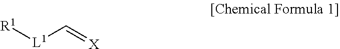

herein in Chemical Formula 1, each substituent is the same as defined in the detailed description.

### CROSS REFERENCE TO RELATED APPLICATIONS

This application is a National Stage of International Application No. PCT/KR2023/004141 filed Mar. 29, 2023, claiming priority based on Korean Patent Application No. 10-2022-0039137 field Mar. 29, 2022 and Korean Patent Application No. 10-2023-0040647 filed Mar. 28, 2023.

### TECHNICAL FIELD

This invention relates to a brightening composition and a skin brightening method using the same.

### BACKGROUND ART

A skin performs various functions essential for the human body to survive. Barrier functions to maintain homeostasis inside the human body in response to environmental changes, sensory functions to recognize external changes, and body temperature control functions are among the most representative skin functions. Among the various functions of the skin, in particular, the barrier functions of the skin are mainly manifested by the stratum corneum at the outermost portion of the skin. Since the stratum corneum has been reported to affect functions, roles, structures, and the like of an inner living cell layer, that is, an epidermal layer or a dermal layer, as well as performs the simple barrier functions, its importance is constantly increasing. This stratum corneum is composed of dead keratinocytes and intercellular lipids, and plays a key function as a skin protective layer that protects the skin from external stimuli and prevents moisture from evaporating from the inside. In addition, the keratinocytes in the stratum corneum create a skin barrier through differentiation and keratinization processes.

There are various factors that cause aging in human skin. In particular, ultraviolet (UV) rays cause wrinkles, a decrease in elasticity, pigmentation, and a decrease in a skin moisture content due to damage on the skin barrier. When the skin surface moisture content decreases due to skin damage caused by the ultraviolet rays, the stratum corneum on the skin surface loses flexibility and thus makes the skin dry, eventually failing in properly functioning as a barrier. Therefore, in order to enhance the skin barrier, it is very important to maintain moisturizing power of the skin.

However, as aging progresses, the skin barrier weakens and it becomes difficult to maintain the skin's moisture content, and thus recently, active research is being conducted on methods to whiten the skin while increasing the skin's defense power regardless of the skin's moisture content, and in particular, research is being actively conducted on melanocytes that are closely related to skin pigmentation.

### DISCLOSURE

### Technical Problem

An embodiment provides a cosmetic composition for skin brightening.

Another embodiment provides a skin brightening method of a subject, which includes administering the cosmetic composition for skin brightening to a subject.

### Technical Solution

According to an embodiment, provided is a brightening composition which includes a compound represented by Chemical Formula 1 as an active ingredient.

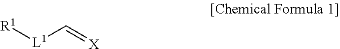

In Chemical Formula 1,
X is CRR′ (R and R′ are independently a hydrogen atom or a substituted or unsubstituted C1 to C10 alkyl group), an oxygen atom, or a sulfur atom,
L¹ is a substituted or unsubstituted C1 to C20 alkylene group, and
R¹ is a hydroxy group, a substituted or substituted C1 to C20 alkyl group, or a substituted or unsubstituted C1 to C20 alkoxy group.

In Chemical Formula 1, X may be an oxygen atom and R¹ may be a hydroxy group.

Chemical Formula 1 may be represented by Chemical Formula 1-1.

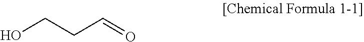

The compound represented by the above chemical formula 1 may be included in a concentration of 0.01 μM to 10 mM based on a total amount of the composition.

The compound represented by Chemical Formula 1 may be derived from lactobacillus ferment.

The above brightening composition may be a cosmetic composition.

According to another embodiment, provided is a skin brightening method of a subject, which includes administering a cosmetic composition including the compound represented by Chemical Formula 1 as an active ingredient to a subject.

According to another embodiment, a use of the compound represented by Chemical Formula 1 for the preparation of a brightening composition is provided.

According to another embodiment, a skin brightening method is provided by applying to the skin a composition including an effective amount of the compound represented by Chemical Formula 1.

The compound and concentration represented by Chemical Formula 1 may be as described above.

### Advantageous Effects

A composition according to an embodiment can achieve a skin brightening effect by inhibiting melanin production of melanocytes in human skin without cytotoxicity, thereby suppressing hyperpigmentation of the skin.

### DESCRIPTION OF THE DRAWINGS

FIG. 1 is a photograph showing that a composition according to an embodiment inhibits melanin production in human normal melanocytes. (Photograph of a cell suspension obtained by collecting cells treated with a test substance and cultured for 8 days in an e-tube and dissolving them in a 1N NaOH solution)

FIG. 2 is a graph showing that a composition according to an embodiment inhibits melanin production in human normal melanocytes.

FIG. 3 is a graph showing that a composition according to an embodiment does not cause cytotoxicity.

### BEST MODE

Hereinafter, embodiments of the present invention will be described in detail so that those skilled in the art to which the present invention pertains could easily practice. However, this disclosure may be embodied in many different forms and is not construed as limited to the example embodiments set forth herein.

In this specification, improvement in skin brightening function means that the color of the stratum corneum located on the outer surface of the skin changes to bright and white. The stratum corneum, which is a primary barrier of the skin, may be easily damaged by an external environment. In general, an external preparation may be prescribed in order to impart brightening power to the skin, which is a temporary solution but not a fundamental solution to the problem, and furthermore, since the external preparation rather solves most of problems but has an effect of just covering black marks on the skin, the hyperpigmentation problem of the skin has not been fundamentally improved through the prescription of simply applying the external preparation to the skin. Accordingly, the present inventors have confirmed that a compound represented by a specific chemical formula, which constitutes a composition according to an embodiment, solves the fundamental problem and thus fundamentally improve skin brightening efficacy, completing the present invention.

More specifically, the composition according to an embodiment can be expected to have a skin brightening effect by inhibiting human normal melanocytes from synthesizing melanin.

In this regard, it has been revealed that melanin production increases when the intracellular concentration of c-AMP increases, and melanin production decreases when the extracellular signal regulated kinase (ERK) pathway is activated. However, as a method for activating the extracellular signal regulated kinase pathway, a method of stimulating a signal transduction pathway that phosphorylates extracellular signal-regulated kinase is well known, and recently, when protein phosphatase 2A (PP2A), an enzyme that dephosphorylates extracellular signal-regulated kinase, is inhibited, it is expected that the extracellular signal-regulated kinase will be activated, ultimately achieving a skin brightening effect through the decomposition of microphthalmia-associated transcription factor (MITF), but an effective method for inhibiting protein phosphatase 2A has not been discovered.

In this regard, the inventors of the present invention have completed the present invention by confirming that there is a melanin synthesis inhibitory effect when treating human normal melanocytes with a composition including a specific compound as an active ingredient (more specifically, a composition containing the specific compound as an active ingredient in a specific concentration range).

On the other hand, in the conventional methods, substances directly inhibiting the melanin-producing enzymes (TRP1, TRP2, tyrosinase) should be used at a high concentration and may not only exhibit side effects such as skin irritation and the like, but also kojic acid has been banned because of the potential to cause skin cancer. However, the composition according to an embodiment is not a substance directly inhibiting the melanin-producing enzymes (TRP1, TRP2, tyrosinase) and thus may not only irritate the skin and thus has almost no side effects, while exhibiting a much safer and more powerful brightening effect through inhibition of melanin production.

In the present specification, when a definition is not otherwise provided, the term “combination” refers to mixing or copolymerization. In addition, “copolymerization” refers to block copolymerization or random copolymerization, and “copolymer” refers to a block copolymer or a random copolymer.

A skin brightening composition according to an embodiment of the present invention is described below.

A skin brightening composition according to an embodiment of the present invention includes a compound represented by Chemical Formula 1 as an active ingredient.

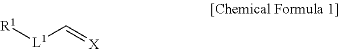

In Chemical Formula 1,
X is CRR′ (R and R′ are independently a hydrogen atom or a substituted or unsubstituted C1 to C10 alkyl group), an oxygen atom, or a sulfur atom,
L¹ is a substituted or unsubstituted C1 to C20 alkylene group, and
R¹ is a hydroxy group, a substituted or substituted C1 to C20 alkyl group, or a substituted or unsubstituted C1 to C20 alkoxy group.

For example, the compound represented by Chemical Formula 1 may be a material derived from lactobacillus ferment. As the need for skin immunity has increased due to the recent increase in interest in health, efforts are being made to develop microbiome materials that enhance a skin's defense function and have various skin benefits, but there are still few materials that can be expected to have a skin brightening effect in this regard. However, the inventors of the present invention started with an idea that the microbiome materials might be a source capable of achieving the skin brightening effect and have conducted research and experiments on a wide variety of microbiome materials for several years and confirmed that the skin brightening effect could be achieved by using a compound represented by Chemical Formula 1 derived from lactobacillus ferment, completing the present invention.

For example, in Chemical Formula 1, X may be an oxygen atom and R¹ may be a hydroxy group.

For example, Chemical Formula 1 may be represented by Chemical Formula 1-1, but is not necessarily limited thereto.

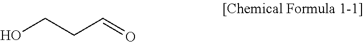

For example, the compound represented by Chemical Formula 1 may be included in a concentration of 0.01 μM to 10 mM based on a total amount of the composition. When the compound represented by Chemical Formula 1 is included in the above concentration range, the brightening effect may be further doubled by effectively inhibiting melanin production of melanocytes of normal human skin without cytotoxicity.

Specifically, the compound represented by Chemical Formula 1 in the composition may be included at a concentration of 0.01 μM to 10 mM, for example, a concentration of 0.1 μM to 10 mM, for example, a concentration of 1 μM to 10 mM, for example, a concentration of 0.01 mM to 10 mM, for example, a concentration of 0.1 mM to 10 mM, based on a total amount of the composition.

For example, the compound represented by Chemical Formula 1 in the composition may be included at a concentration of 0.01 μM or more, for example, 0.1 μM or more, for example, 1 μM or more, for example, 10 μM or more, for example, 100 μM or more, and may be included at a concentration of 10 mM or less, for example, 1 mM or less, based on a total amount of the composition.

When the compound represented by Chemical Formula 1 is used at a concentration of less than 0.01 μM, a degree of inhibition of new melanin production in melanocytes is minimal, and thus the effect of improving skin brightening function cannot be achieved. In addition, when the compound represented by Chemical Formula 1 is used at a concentration exceeding 10 mM, it may be undesirable as it may exhibit cytotoxicity and cause harm to the human body. Furthermore, when the compound represented by Chemical Formula 1 is a compound derived from lactobacillus ferment, the compound represented by Chemical Formula 1 (derived from lactobacillus ferment) in the composition may be included at a concentration of 10 μM to 10 mM, for example, 100 μM to 5 mM. Compared with a case when the compound represented by Chemical Formula 1 is separately synthesized and used, when the compound represented by Chemical Formula 1 is used in the form of lactobacillus ferment, which corresponds to a case of using a natural product, its concentration ranges for inhibiting new melanin production in melanocytes without cytotoxicity may be different from the above ranges. This is thought to be because unlike chemical compounds, natural products are limited or changed in expressing desired functions by interactions with various component included in a composition.

Hyperpigmentation on the skin may be caused by various factors such as hormonal abnormalities after inflammation reaction of the skin, genetic diseases, ultraviolet (UV) irradiation, and the like, and among them, abnormality in synthesis and distribution of melanin pigments and the like may be counted as main factors.

Melanin has a main function of scavenging oxygen radicals and protecting the skin from damages caused therefrom. Therefore, a lot of the melanin means an effective countermeasure system for protecting the skin from physical and chemical toxic substances. The melanin is produced by enzymatic action and spontaneous oxidation after tyrosine is converted into dopaquinone by the melanin-producing enzymes (TRP1, TRP2, tyrosinase) in melanocytes.

Methods for inhibiting the melanogenesis known up to date are largely as follows.

First, there is a method of blocking ultraviolet (UV) to remove a main cause of the melanogenesis. In this method, a light scattering agent or a light blocking agent may be included in a cosmetic composition, which brings about good results.

Next, the melanogenesis may be suppressed by inhibiting synthesis of core carbohydrates required for activity of the melanin-producing enzymes (TRP1, TRP2, tyrosinase), for example, glucosamine.

In addition, kojic acid or arbutin may be used to inhibit a function of the melanin-producing enzyme (TRP1) involved in the melanogenesis.

Furthermore, substances with specific toxicity to the melanocytes producing melanin, for example, hydroquinone may be used to interfere division of the melanocytes.

In addition, a method of reducing and decolorizing the produced melanin has been introduced.

Currently, most of research for discovering a brightening composition is focused on substances that directly inhibit the melanin-producing enzymes (TRP1, TRP2, tyrosinase) involved in the melanin synthesis. However, in addition to directly inhibiting the melanin-producing enzymes (TRP1, TRP2, tyrosinase), it has been suggested that brightening effects may be expected by regulating signal transduction involved in expressions of the melanin-producing enzymes (TRP1, TRP2, tyrosinase). (Briganti S, Camera E, Picardo M. Pigment Cell Res, 2003, 16 2:101-10)

A composition according to an embodiment of the present invention is particularly effective in inhibiting the new production of melanin in melanocytes, and therefore has an excellent skin brightening effect when used on skin where hyperpigmentation is induced by a skin pigmentation inducer.

An embodiment provides a composition for skin brightening including the compound represented by Chemical Formula 1 as an active ingredient, wherein the composition may include a pharmaceutically effective amount of the compound represented by Chemical Formula 1 alone or at least one pharmaceutically acceptable carrier, excipient, or diluent.

In the above, “pharmaceutically effective amount” refers to an amount sufficient to allow the physiologically active ingredient to be administered to an animal or human to exhibit desired physiological or pharmacological activity. However, the pharmaceutically effective amount may vary according to the degrees of symptoms, ages, weights, health status, sexes, administration routes, and duration of treatment.

In addition, “pharmaceutically acceptable” refers to physiologically acceptable when administered to humans, and usually does not cause allergic reactions or similar reactions, such as gastrointestinal disorders or dizziness. Examples of the carrier, excipient, and diluent may include lactose, dextrose, sucrose, sorbitol, mannitol, xylitol, erythritol, maltitol, starch, acacia rubber, alginate, gelatin, calcium phosphate, calcium silicate, cellulose, methyl cellulose, polyvinylpyrrolidone, water, methylhydroxybenzoate, propylhydroxybenzoate, talc, magnesium stearate, and mineral oils. In addition, it may further include fillers, anti-coagulants, lubricants, wetting agents, fragrances, emulsifiers, and antiseptics.

For example, the composition may be a cosmetic composition.

In the present specification “cosmetic” may refer to any material that may have a medical function in addition to the cosmetic function.

The formulation of the cosmetic composition is not particularly limited and may be appropriately selected as desired.

For example, the cosmetic composition may be formulated into formulations such as solutions, suspension liquids, emulsions, pastes, gels, creams, lotions, powders, soaps, surfactant-containing cleansings, oils, powder foundations, emulsion foundations, wax foundations, and sprays, but is not limited thereto. More specifically, it may be formulated into cosmetic compositions such as detergents, tonics, hair dressings, nourishing lotions, essences, serums, treatments, conditioners, shampoos, lotions, wools, or hair dyes, and the like, and may be formulated into basic cosmetics such as an oil-in-water (O/W) type, a water-in-oil (W/O), and the like. For example, the composition may have a formulation selected from skin lotion, skin toner, astringent, lotion, milk lotion, moisture lotion, nourishing lotion, massage cream, nourishing cream, moisture cream, hand cream, ointment, foundation, essence, nourishing essence, pack, soap, cleansing foam, cleansing lotion, cleansing cream, body lotion, body cleanser, gel, cream, patch, or spray. In addition, in the composition, in addition to the above-mentioned essential components in each formulation, other components may be appropriately selected and formulated without difficulty by a person of ordinary skill in the art according to types or use purposes of other external preparations. For example, ultraviolet (UV) blocking agents, hair conditioning agents, fragrances, and the like may be further included.

The cosmetic composition may include a cosmetically acceptable medium or base. These are all formulations suitable for topical applications. The cosmetic composition may be provided in the form of emulsions obtained by dispersing an oil phase in an aqueous phase, suspensions, microemulsions, microcapsules, microgranules, or ion-type (liposome) and/or non-ionized vesicle dispersing agents, or in the form of creams, skins, lotions, powders, ointments, sprays, or conceal sticks. These compositions may be prepared according to conventional methods in the art.

When the formulation of the present invention is a solution or emulsion, a solvent, a solubilizer, or an emulsifier may be used as carrier components. For example, water, ethanol, isopropanol, ethyl carbonate, ethyl acetate, benzyl alcohol, benzyl benzoate, propylene glycol, 1,3-butylglycol oil, glycerol aliphatic ester, polyethylene glycol, or fatty acid ester of sorbitan may be used.

If the formulation of the present invention is a suspension, the carrier component may be a diluent of a liquid such as water, ethanol or propylene glycol, a suspending agent such as ethoxylated isostearyl alcohol, polyoxyethylene sorbitol ester, and polyoxyethylene sorbitan ester, microcrystalline cellulose, aluminum metahydroxide, bentonite, agar, tragacanth, and the like.

If the formulation of the present invention is pastes, creams, or gels, the carrier component may be animal oil, vegetable oil, wax, paraffin, starch, tragacanth, cellulose derivatives, polyethylene glycol, silicone, bentonite, silica, talc or zinc oxide.

If the formulation of the present invention is powders or sprays, the carrier component may be lactose, talc, silica, aluminum hydroxide, calcium silicate, or polyamide powders. Particularly, in the case of sprays, a propellant such as a chlorofluorohydrocarbon, propane/butane, or dimethyl ether may be additionally included.

In an embodiment of the present invention, the cosmetic composition may include thickeners. The thickeners included in the cosmetic composition of the present invention may be methyl cellulose, carboxyl methyl cellulose, carboxyl methyl hydroxy guanine, hydroxy methyl cellulose, hydroxyethyl cellulose, a carboxyl vinyl polymer, polyquaternium, cetearyl alcohol, stearic acid, and carrageenan, preferably one or more of carboxyl methyl cellulose, a carboxyl vinyl polymer, and polyquaternium may be used, and more preferably a carboxyl vinyl polymer may be used.

In an embodiment of the present invention, the cosmetic composition may include a variety of suitable bases and additives as needed, and the types and amounts of these components may be easily selected by the inventor. If necessary, it may include an acceptable additive, and may further include for example, conventional ingredients such as antiseptics, pigments, additives, and the like.

The antiseptics may specifically be phenoxyethanol or 1,2-hexanediol, and the fragrances may be artificial fragrances.

In an embodiment of the present invention, the cosmetic composition may include a composition selected from a water-soluble vitamin, an oil-soluble vitamin, a polymeric peptide, a polymeric polysaccharide, a sphingolipid, and a seaweed extract. Other ingredients that may be added include fats and oils, humectants, emollients, surfactants, organic and inorganic pigments, organic powders, ultraviolet (UV) absorbers, antiseptics, fungicides, antioxidants, plant extracts, pH adjusters, alcohols, pigments, fragrances, blood circulation accelerators, coolants, anhidrotics, purified water, and the like.

In addition, the compounding components which may be added other than these are not limited thereto. Moreover, any component may be blended in the range which does not damage the purpose and effect of the invention.

Furthermore, the cosmetic composition according to an embodiment may be used not only as a pharmaceutical composition as described above, but also as a dietary supplement. For example, it may be easily used as main ingredients, auxiliary ingredients, food ingredients, food additives, functional foods, or beverages.

The “food” means a natural or processed product including one or more nutrients, and preferably means that it is ready to be eaten directly after a certain amount of processing. It includes all foods, food additives, functional foods, and beverages.

The foods to which the food composition can be added may include, for example, various foods, beverages, gums, teas, vitamin composites, and functional foods. In addition, the foods may include special nutritional products (e.g., formulas, baby food, etc.), processed meat products, fish products, tofu, jellies, noodles (e.g. ramen noodles, etc.), breads, dietary supplements, seasoned foods (e.g., soy sauce, soybean paste, red pepper paste, mixed soy sauce, etc.), sauces, sweets (e.g. snacks), candy, chocolate, gum, ice cream, dairy products (e.g. fermented milk, cheese, etc.), other processed foods, kimchi, pickles (various kimchis, pickles, etc.), beverages (e.g., fruit beverages, vegetable beverages, soy milk, fermented beverages, etc.), natural seasonings (e.g., ramen soup, etc.), but are not limited thereto. The foods, beverages, or food additives may be prepared by conventional manufacturing methods.

In addition, “functional foods” or “health functional foods” refers to a food group that has added values to foods by using physical, biochemical, or biotechnological techniques to act and express functions of foods for specific purposes, or foods that are processed and designed to fully express the body's regulatory functions, such as defense rhythm control of food compositions, disease prevention, and recovery of living bodies. It may specifically be health functional foods. The functional foods may include acceptable food auxiliary additives and may further include suitable carriers, excipients, and diluents commonly used in the manufacture of functional foods.

The types of dietary supplements are not limited thereto, but may be in a form of powders, granules, tablets, capsules, or beverages.

Another embodiment provides a skin brightening method of a subject including administering the composition to the subject. The composition is the same as described above.

The administrating may be performed by a method known in the art. The administrating may be performed directly to the subject by any means, for example, by routes such as intravenous, intramuscular, oral, transdermal, mucosal, intranasal, intratracheal or subcutaneous administration. The administrating may be systemically or locally. The administrating may be topically performed to a site where skin aging is present. The administrating may be, for example, performed by application. The application refers to any method of bringing the composition into contact with the skin of an individual in an appropriate way, through which the composition may be absorbed into the skin.

The subject may be a mammal, such as a human, cow, horse, pig, dog, sheep, goat, or cat. The subject may be a subject in need of ameliorating, delaying, or inhibiting skin hyperpigmentation. The subject may be an individual in need of inhibiting cell death, inhibiting protein expression of melanin-producing enzyme (tyrosinase), or increasing antioxidant activity.

In the administrating, the composition according to an embodiment may be administered in an amount of about 0.01 mg to about 10,000 mg, about 0.1 mg to about 1,000 mg, for example, about 0.1 mg to about 500 mg, about 0.1 mg to about 100 mg, about 0.1 mg to about 50 mg, about 0.1 mg to about 25 mg, about 1 mg to about 1,000 mg, about 1 mg to about 500 mg, about 1 mg to about 100 mg, about 1 mg to about 50 mg, about 1 mg to about 25 mg, about 5 mg to about 1,000 mg, about 5 mg to about 500 mg, about 5 mg to about 100 mg, about 5 mg to about 50 mg, about 5 mg to about 25 mg, about 10 mg to about 1,000 mg, about 10 mg to about 500 mg, about 10 mg to about 100 mg, about 10 mg to about 50 mg, or about 10 mg to about 25 mg per day. Or the composition according to an embodiment may be administrated so that the composition may work at a concentration (working concentration) of about 0.01 μM to about 25 μM, about 0.05 μM to about 5 μM, about 0.075 μM to about 3.75 μM, about 0.1 μM to about 2.5 M, about 0.15 μM to about 2 μM, about 0.25 μM to about 1.5 μM, about 0.4 μM to about 1.25 μM, about 0.5 μM to about 1.2 μM, or about 0.75 μM to about 1.15 μM.

### Detailed Description of the Embodiments

Advantages and features of the present invention and methods for achieving them will be apparent with reference to the examples described in detail below. The present invention will be described in detail with reference to examples. However, these examples are specifically provided for describing the present invention, and the range of the present invention is not limited to these examples.

### EXAMPLE

### Experimental Example 1

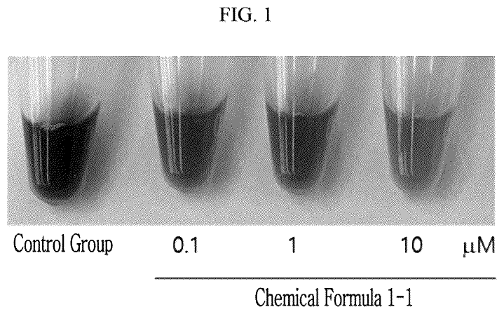

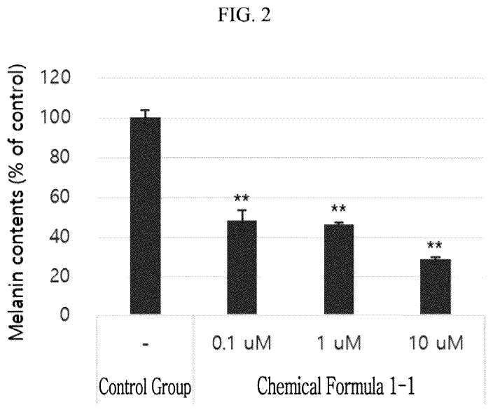

Melanin-producing cells isolated from normal human skin (normal human epidermal melanocytes) were added at each final concentration of 0 μM (a control group), 0.1 μM, 1 μM, and 10 μM to a culture medium prepared by dissolving a compound represented by Chemical Formula 1-1 (Chr. Hansen A/S) in DMSO at 1000×. The cells were cultured in an incubator under 5% CO₂ at 37° C. up to 8 days by changing the culture medium containing the experiment material every two days. After the experiment, the cells were dissolved in 1N NaOH and then, photographed to measure absorbance at 475 nm and compare relative amounts of melanin, and the results are shown in FIGS. 1 and 2. (** statistically significant difference P value vs. control group <0.01)

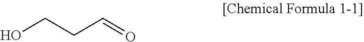

Referring to FIGS. 1 and 2, when the compound represented by Chemical Formula 1-1 was included, it was confirmed that melanin production was effectively suppressed in the human normal melanocytes. Particularly, the melanin production was suppressed depending on a concentration of the compound represented by Chemical Formula 1-1.

### Experimental Example 2

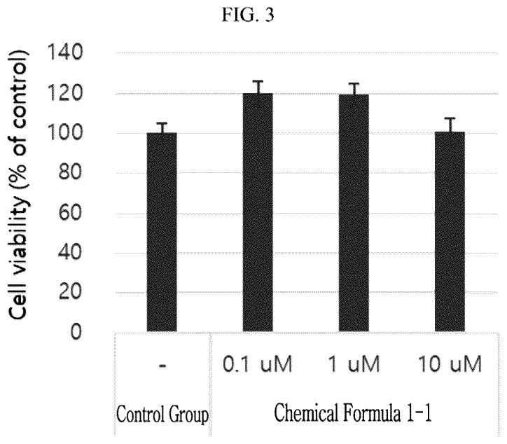

Melanin-producing cells isolated from normal human skin (normal human epidermal melanocytes) were added at each final concentration of 0 μM (a control group), 0.1 μM, 1 μM, and 10 μM to a culture medium prepared by dissolving a compound represented by Chemical Formula 1-1 (Chr. Hansen A/S) in DMSO at 1000×. The cells were cultured in an incubator under 5% CO₂ at 37° C. up to 8 days by changing the culture medium containing the experiment material every two days. After the experiment, the medium was washed away, and a new medium containing CCK8 (a cell counting kit, Sigma Aldrich Co., Ltd.) was added to the cells, which were additionally cultured for 1 hour. After the additional culture, the medium was collected to measure absorbance at 450 nm and 650 nm and then, subtract the absorbance at 650 nm from the absorbance at 450 nm, which was compared with that of the control group to compare cell viability, and the results are shown in FIG. 3.

Referring to FIG. 3, when the compound represented by Chemical Formula 1-1 was included, there was no cytotoxicity, compared with the control group.

Referring to FIGS. 1 to 3, when the compound represented by Chemical Formula 1-1 (Chr.Hansen A/S) was included at each concentration of 0.1 μM, 1 μM, and 10 μM, it was all confirmed to have an inhibitory effect on melanin production without cytotoxicity. In general, considering that if a concentration of an active component is confirmed in a cell experiment, the active component used in the cell experiment is actually 100× to 1000× of the concentration in a composition, the compound represented by Chemical Formula 1, which is an active component in a brightening composition according to an embodiment, may be included at a concentration of 10 μM to 10 mM based on a total amount of the composition, which may be the most optimal amount for implementing the inhibitory effect on melanin production without cytotoxicity.

Although the preferred embodiments of the present invention have been described in detail, the scope of the present invention is not limited thereto, and various modifications and improvements by those skilled in the art using the basic concept of the present invention defined in the following claims are also within the scope of the invention.

1. A composition, comprising a compound of the following Chemical Formula 1 as an active ingredient: 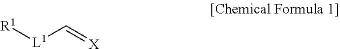
wherein, in Chemical Formula 1,
X is CRR′ (R and R′ are independently a hydrogen atom or a substituted or unsubstituted C1 to C10 alkyl group), an oxygen atom, or a sulfur atom,
L¹ is a substituted or unsubstituted C1 to C20 alkylene group, and
R¹ is a hydroxy group, a substituted or substituted C1 to C20 alkyl group, or a substituted or unsubstituted C1 to C20 alkoxy group.

2. The composition of claim 1, wherein
in Chemical Formula 1,
X is an oxygen atom and R¹ is a hydroxy group.

3. The composition of claim 1, wherein the compound of Chemical Formula 1 is a compound of the following Chemical Formula 1-1:

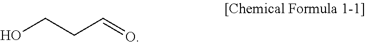

4. The composition of claim 1, wherein
the compound represented by Chemical Formula 1 is included in a concentration of 0.01 μM to 10 mM based on a total amount of the composition.

5. The composition of claim 1, wherein
the compound of Chemical Formula 1 is obtained from lactobacillus ferment.

6. The composition of claim 1, wherein
the composition is a brightening cosmetic composition.

7. A skin brightening method of a subject, applying an effective amount of the composition of claim 1 to the skin.

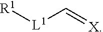

8. The skin brightening method of claim 7, wherein
in Chemical Formula 1,
X is an oxygen atom and R¹ is a hydroxy group.

9. The skin brightening method of claim 7, wherein
the compound of Chemical Formula 1 is a compound of the following Chemical Formula 1-1:

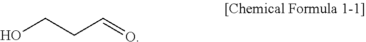

10. The skin brightening method of claim 7, wherein
the compound of Chemical Formula 1 is included in a concentration of 0.01 μM to 10 mM based on a total amount of the composition.
